(19) United States
(12) Patent Application Publication
(10) Pub. No.: US 2006/0240496 A1
(43) Pub. Date: Oct. 26, 2006

(54) Immunogens, derivatives and immunoassay for ethyl glucuronide

(76) Inventors: Lakshmi Anne, Hayward, CA (US); Vani Bodepudi, San Ramon, CA (US)

Correspondence Address: Jonathan M. Benns;WORKMAN NYDEGGER, 1000 Eagle Gate Tower, 60 East South Temple, Salt Lake City, UT, 84111, US

(21) Appl. No.: 11/407,415

(22) Filed: Apr. 20, 2006

Related U.S. Application Data
(60) Provisional application No. 60/673,697, filed on Apr. 21, 2005.

(51) Int. Cl. G01N 33/53; C07K 16/18; C07K 14/47

(52) U.S. Cl. 435/7.92; 530/387.1; 530/395; 536/53

(57) ABSTRACT

Ethyl glucuronide (“EtG”) analogs can be prepared for use as immunodiagnostic reagents and in immunodiagnostic protocols. The EtG analogs can be in the form of EtG-based immunogens, and EtG-based antigens. The EtG-based immunogens can be used for preparing anti-EtG antibodies, which can be used in immunoassays. Accordingly, improved immunoassay techniques for detecting EtG can be performed with the EtG-based antigens, and anti-EtG antibodies prepared from EtG-based immunogens.

### CROSS-REFERENCE TO RELATED APPLICATIONS

This U.S. Patent Application claims benefit of U.S. Provisional Ser. No. 60/673,697, filed on Apr. 21, 2005, which is incorporated herein by reference.

### BACKGROUND OF THE INVENTION

1. The Field of the Invention

The present invention relates to ethyl glucuronide and ethyl glucuronide analogs for use as immunodiagnostic reagents and in immunodiagnostic protocols. More particularly, the present invention relates to ethyl glucuronide and -ethyl glucuronide analogs, ethyl glucuronide-based immunogens, ethyl glucuronide-based antigens, antibodies prepared from ethyl glucuronide-based immunogens, and methods of making and using the same.

2. The Related Technology

Ethyl glucuronide (“EtG”), which is shown in FIG. 1, is a direct metabolite of ethyl alcohol formed by the conjugation of ethanol with activated glucuronic acid in the presence of UDP glucuronyl transferase on mitochondrial membranes. While ethanol is detectable for only a few hours after consumption, EtG is detectable for up to four or five days after alcohol consumption, making it a reliable target analyte for determining alcohol consumption. Monitoring of alcohol consumption is important for many reasons including use in conjunction with safety-sensitive programs such as airline pilots and health care or emergency care professionals.

Currently, EtG is detected by gas chromatography/mass spectrometry (“GC/MS”) and liquid chromatography/tandem mass spectrometry (“LC/MS/MS”). Such methods are time-consuming and expensive. Zimmer et al. (J. Analytical Toxicology, 26:11-16, 2002; incorporated herein in its entirety) describes an enzyme linked immunoassay (“ELISA”) test to detect EtG using a polyclonal antiserum induced by immunization with EtG linked directly to peroxidase enzyme via the carboxyl group at position 5 of EtG. The use of polyclonal antibodies can result in an increased number of false positives (23.2%) and false negatives (24.3%), which indicates poor specificity, and sensitivity of the assay. In 2001, Mediagnost Company Ltd. (Reutlingen, Germany) began development of an ELISA test utilizing monoclonal antibodies prepared by immunization with a lipopeptide conjugate of EtG (Wurst et al., Addiction 98(Suppl. 2), pages 51-61, 2003). However, to date, no product has been made available.

Immunoassays are becoming increasingly popular as methods for detecting or monitoring the presence of drugs or analytes in body fluids or biological samples. A particular challenge in the development of immunoassays is the production of an antibody to the target drug/analyte since many are not inherently antigenic. Generally, the drug must be modified to make an antigenic derivative, yet the antibody produced to the antigenic derivative must be able to recognize the drug as it is contained in the fluid specimen to be tested with an appropriately useful degree of sensitivity, generally a level with physiological and/or pharmacological significance. Sensitivity is not the only concern. Often a variety of metabolites exist and other drugs may be present along with the target analyte. Preferably, an antibody to a particular drug or metabolite has minimal, if any, cross-reactivity with other metabolites or other drugs.

Some immunoassay techniques have been developed to detect various chemicals in biological samples and are well-suited for such commercial analytical applications. Accordingly, immunoassays can be used to quickly determine the amount of a chemical and/or chemical metabolite in a person's blood. Examples of immunoassays can include, but are not limited to, homogeneous microparticle immunoassay (e.g., immunoturbidimetric) or quantitative microsphere systems (“QMS®”), fluorescence polarization immunoassay (“FPIA”), cloned enzyme donor immunoassay (“CEDIA”), chemiluminescent microparticle immunoassay (“CMIA”), enzyme multiplied immunoassay technique (“EMIT”), and the like.

Therefore, it would be advantageous to have improved EtG analogs, EtG-based immunogens, and EtG-based antigens prepared from EtG analogs, antibodies prepared from EtG-based immunogens, and methods of making and using the same. Additionally, it would be advantageous to have improved immunoassay techniques that can be used with the EtG-based immunogens, EtG-based antigens, and antibodies prepared from EtG-based immunogens prepared in accordance with the present invention. The current invention will have better sensitivity and specificity with the use of monoclonal antibody compared to prior assays. Good sensitivity and especially specificity in the screening immunoassay are important because of the cost involved in confirming the false positives.

### SUMMARY OF THE INVENTION

Embodiments of the present invention can include EtG analogs, such as EtG-based immunogens and EtG-based immunoassay reagents, for use as immunodiagnostic reagents and in immunodiagnostic protocols. Also, embodiments of the present invention can include improved immunoassay techniques that can be used with the EtG-based immunogens, EtG-based immunoassay reagents, and anti-EtG antibodies prepared from EtG-based immunogens prepared in accordance with the present invention.

One embodiment of the present invention can be an EtG analog for use in a process for preparing and/or implementing an immunoassay for detecting EtG in a sample. Such an EtG analog can be prepared in accordance with Formulas 1A, 1B, 2A, 2B, 2C, 2D, 3A, 3B and/or 3C shown below. In accordance with the formulas, the EtG analog can be characterized as follows: n can be greater than or equal to 1 and/or less than about 1000; L can be at least one of the groups O, S, CO, COO, SO₂, CH₂, NH, NH(CH₂)₂NH, CONH, Ph, NHCH₂Ph, or the like; X can be at least one of a bond between L and Y, an aromatic group, or an aliphatic group; Y can be selected from the group consisting of aliphatic, alcohol, amine, amide, carboxylic acid, aldehyde, ester, activated ester, aliphatic ester, imidoester, isocyanate, isothiocyanate, anhydride, thiol, thiolactone, diazonium, maleimido, NHS, O—NHS, and a linker derived therefrom coupled with an operative moiety; and Z can be an operative moiety.

In one embodiment, X can be at least one of a bond between L and Y, a substituted or unsubstituted aromatic or aliphatic group having from 1 to 2 rings, or a saturated or unsaturated, substituted or unsubstituted, and straight or branched chain having from 1 to 20 carbon and/or hetero chain atoms. Also, when used, the operative moiety Z can be selected from the group consisting of proteins, lipoproteins, glycoproteins, polypeptides, poly(amino acids), polysaccharides, nucleic acids, polynucleotides, teichoic acids, detectable labels, radioactive isotopes, enzymes, enzyme fragments, enzyme donor fragments, enzyme acceptor fragments, enzyme substrates, enzyme inhibitors, coenzymes, fluorescent moieties, phosphorescent moieties, anti-stokes up-regulating moieties, chemiluminescent moieties, luminescent moieties, dyes, sensitizers, particles, microparticles, magnetic particles, solid supports, liposomes, ligands, receptors, hapten radioactive isotopes, albumin, human serum albumin, bovine serum albumin, keyhole limpet hemocyanin, and combinations thereof. In the instance the analog is an immunogen, Z can be at least one of the following: human serum albumin with n being about 1 to about 35; bovine serum albumin with n being about 1 to about 35; or keyhole limpet hemocyanin with n being about 1 to about 500. In the instance the analog is an immunoassay reagent for detecting EtG, Z can be a detectable label. For example, the detectable label can be an enzyme (e.g., Glucose-6-phosphate dehydrogenase “G6PDH”), enzyme fragment, or enzyme donor fragment (e.g., β-galactosidase enzyme donor fragment ED28).

In one embodiment, the present invention can include an antibody prepared with an EtG-based immunogen, wherein the antibody is an anti-EtG antibody capable of interacting with EtG and the EtG analog. Also, the antibody can be capable of interacting with EtG in a sample at a concentration of less than or equal to about 0.05 mg/dL, and have a cross-reactivity of less than about 1% with at least one of methyl glucuronide, lorazepam glucuronide, oxazepam glucuronide, temazepam flucuronide, D-glucose, 1-butanol, or 2-butanol.

In one embodiment, the present invention can include an immunoassay system for detecting EtG, wherein the system can have an anti-EtG antibody prepared with an EtG-based immunogen described herein. Also, the immunoassay system can have an EtG-based immunoassay reagent.

In one embodiment, the present invention can include a method of detecting EtG in a sample. Such a method can include the following: obtaining a sample from a subject suspected of consuming ethyl alcohol; combining an anti-EtG antibody and an EtG analog with the sample to form a first composition, said antibody and analog being free within the first composition, and said antibody being capable of binding EtG and the EtG analog; allowing any free EtG from the sample and the EtG analog to compete for binding with the antibody; and detecting binding between the EtG analog and the antibody. By being free, the antigen and antibody are capable of freely moving within a solution so as to be solubilized or suspended, rather than being attached to the reaction vessel as in ELISA assays. The anti-EtG antibody can be prepared with an EtG-based immunogen as described herein. The EtG analog can be prepared as described herein to include a detectable label, such as an enzyme (e.g., G6PDH), enzyme fragment or enzyme donor fragment (e.g., the β-galactosidase enzyme donor fragment ED28 and n is about 2).

Additionally, the method of detecting EtG can be a CEDIAN immunoassay. This can include the following: obtaining an EtG analog that includes an enzyme donor; combining an enzyme acceptor with the first composition; combining a substrate with the first composition, wherein the substrate is cleavable by interacting with the enzyme donor and enzyme acceptor; and detecting enzyme activity. Moreover, standardized concentration and/or calibration curves can be prepared with known amounts of EtG so that in addition to detecting the presence of EtG in the sample, the amount or concentration of EtG in the sample can be determined. As such, a method of preparing a concentration and/or calibration curve can include the following: combining a known amount of EtG with the EtG analog and antibody to form a control binding composition; combining an enzyme acceptor with the control binding composition; combining a substrate with the control binding composition, wherein the substrate is cleavable by interacting with the enzyme donor and enzyme acceptor; detecting control enzyme activity; and determining the amount of EtG present in the sample, wherein a comparison between the enzyme activity and the control enzyme activity is an indication of the amount of EtG present in the sample.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings, in which:

FIG. 1 illustrates the chemical structure of ethyl glucuronide.

FIG. 2 shows the chemical structure of an exemplary immunogen prepared from ethyl glucuronide for use in stimulating an immune reaction and antibody production.

FIG. 3 shows the chemical structure of other glucuronide derivatives for forming immunogens for stimulating anti-ethyl glucuronide antibody production or other conjugates useful in detecting ethyl glucuronide.

FIG. 4 shows the chemical structure of a maleimide adduct of ethyl glucuronide for coupling to other molecules, such as enzymes or enzyme fragments.

FIG. 5 is the chemical structure of a maleimide adduct of ethyl glucuronide

coupled to β-galactosidase enzyme donor (ED28).

FIG. 6 is the chemical structure of ethyl glucuronide coupled via a linker to glucose-6-phosphate dehydrogenase.

FIG. 7 is a typical calibration curve for a competitive homogeneous immunoassay for ethyl glucuronide in urine using an Hitachi 917 analyzer.

FIG. 8 is a typical calibration curve for a competitive homogeneous immunoassay for ethyl glucuronide in urine using a Hitachi 717 analyzer.

FIG. 9 is a typical calibration curve for a competitive homogeneous immunoassay for ethyl glucuronide in urine using antibodies from clone 14C5 on an Olympus AU640 analyzer.

FIG. 10 is a typical calibration curve for a competitive homogeneous immunoassay for ethyl glucuronide in urine using antibodies from clone 14C5 on a Hitachi 917 analyzer.

FIG. 11 is a typical calibration curve for a competitive homogeneous immunoassay for ethyl glucuronide in urine using antibodies from clone 12E7 on a Hitachi 917 analyzer.

### DETAILED DESCRIPTION OF THE PREFERRED EMBODIMENTS

Generally, the present invention can include improved EtG analogs for use as immunodiagnostic reagents and in immunodiagnostic protocols. As such, the present invention can include improved EtG analogs, EtG-based immunogens, EtG-based antigens, antibodies prepared from EtG-based immunogens, and methods of making and using the same. More particularly, the present invention can include improved immunoassay techniques that can be used with the EtG-based immunogens, EtG-based antigens, and antibodies prepared from EtG-based immunogens prepared in accordance with the present invention.

In one embodiment, the present invention can include an immunoassay for determining the presence and/or amount of EtG in a sample with suitable specificity and sensitivity, especially one adaptable for use with automated analyzers. Examples of immunoassays for detecting and/or quantifying EtG can include, but are not limited to, homogeneous microparticle immunoassay (e.g., immunoturbidimetric) or quantitative microsphere systems (“QMSO®”), fluorescence polarization immunoassay (“FPIA”), cloned enzyme donor immunoassay (“CEDIA®V”), chemiluminescent microparticle immunoassay (“CMIA”), enzyme multiplied immunoassay technique (“EMIT”), and the like.

In one embodiment of the present invention, EtG can be chemically modified to produce immunogens that are capable of inducing an immunologic response in a mammal so as to produce an anti-EtG antibody. Also, the EtG can be chemically modified to produce antigens that are capable of interacting with the anti-EtG antibodies. Additionally, the EtG can be chemically modified to produce conjugates that include the EtG bound to a label that are also capable of interacting with the anti-EtG antibodies.

In another embodiment of the present invention, an EtG-based immunogen can be used to produce an anti-EtG antibody with specificity and sensitivity for EtG. Also, the anti-EtG antibody directed to EtG can be used in connection with an immunoassay to detect the presence and/or determine the concentration of EtG in a sample, such as a patient specimen. Moreover, the EtG analogs, EtG-based immunogens, EtG-based antigens, and/or anti-EtG antibodies prepared from EtG-based immunogens can be combined in a system or kit for detecting the presence and/or determining the concentration of EtG in samples.

In accordance with one embodiment of the present invention, the methods for making the EtG analogs, EtG-based immunogens, EtG-based antigens, and/or anti-EtG antibodies (e.g., monoclonal and/or polyclonal) prepared from EtG-based immunogens immunogenic derivatives of EtG are described in more detail below. Additionally, immunoassays that can be used with the EtG analogs, EtG-based immunogens, EtG-based antigens, and/or anti-EtG antibodies (e.g., monoclonal and/or polyclonal) prepared from EtG-based immunogens are also described in more detail below.

I. Definitions

Unless stated otherwise, the following terms and phrases have the meanings provided below.

As used herein, the term “affinity” is meant to refer to a measure of the strength of binding between an epitope and an antibody. Accordingly, a single antibody can have a different affinity for various epitopes. This can allow a single antibody to bind strongly to one epitope and less strongly to another. As such, an antibody can have a first affinity to a chemical, such as EtG, and have a second affinity to an EtG analog. However, it is possible for the antibody to have substantially equivalent or similar affinity for both EtG and an EtG analog, which allows the analog to be used to generate antibodies for EtG, and their use in competitive binding studies. Thus, EtG analogs in accordance with the present invention can be used to generate antibodies with affinity for EtG.

As used herein, the term “aliphatic” is meant to refer to a hydrocarbyl moiety, such as an alkyl group, that can be straight or branched, saturated or unsaturated, and/or substituted or unsubstituted, which has twenty or less carbons in the backbone that is used as part of a linker to couple a hapten, such as EtG with an operative group, such as an immunogenic carrier. An aliphatic group may comprise moieties that are linear, branched, cyclic and/or heterocyclic, and contain functional-groups such as ethers, ketones, aldehydes, carboxylates, and the like. Exemplary aliphatic groups include but are not limited to substituted and/or unsubstituted groups of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, alkyl groups of higher number of carbons and the like, as well as 2-methylpropyl, 2-methyl-4-ethylbutyl, 2,4-diethylpropyl, 3-propylbutyl, 2,8-dibutyldecyl, 6,6-dimethyloctyl, 6-propyl-6-butyloctyl, 2-methylbutyl, 2-methylpentyl, 3-methylpentyl, 2-ethylhexyl, and the like. The terms aliphatic or alkyl also encompasses alkenyl groups, such as vinyl, allyl, aralkyl and alkynyl groups.

Substitutions within an aliphatic group can include any atom or group that can be tolerated in the aliphatic moiety, including but not limited to halogens, sulfurs, thiols, thioethers, thioesters, amines (primary, secondary, or tertiary), amides, ethers, esters, alcohols, oxygen, and the like. The aliphatic groups can by way of example also comprise modifications such as azo groups, keto groups, aldehyde groups, carbonyl groups, carboxyl groups, nitro, nitroso or nitrile groups, heterocycles such as imidazole, hydrazino or hydroxylamino groups, isocyanate or cyanate groups, and sulfur containing groups such as sulfoxide, sulfone, sulfide, and disulfide. Additionally, the substitutions can be via single, double, or triple bonds, when relevant or possible.

Further, aliphatic groups may also contain hetero substitutions, which are substitutions of carbon atoms, by hetero atoms such as, for example, nitrogen, oxygen, phosphorous, or sulfur. As such, a linker comprised of a substituted aliphatic can have a backbone comprised of carbon, nitrogen, oxygen, sulfur, phosphorous, and/or the like. Heterocyclic substitutions refer to alkyl rings having one or more hetero atoms. Examples of heterocyclic moieties include but are not limited to morpholino, imidazole, and pyrrolidino.

As used herein, the term “aromatic” is meant to refer to a molecule in which electrons are free to cycle around circular or cyclic arrangements of atoms, which are alternately singly and doubly bonded to one another, and can be used as part of a linker to couple a hapten, such as EtG with an operative group, such as an immunogenic carrier. More properly, these bonds may be seen as a hybrid of a single bond and a double bond, each bond in the ring being identical to every other. Examples of aromatc compounds that can be present in EtG analogs include benzene, benzyl, toluene, xylene, and the like. The aromatic compound can include hetero atoms so as to be a hetero aromatic such as pyridine, furan, tetrahydrofuran, and the like. Also, an aromatic can be a polycyclic aromatic such as naphthalene, anthracene, phenanthrene, polycyclic aromatic hydrocarbons, indole, quinoline, isoquinoline, and the like.

As used herein, the term “amine” is meant to refer to moieties that can be derived directly or indirectly from ammonia by replacing one, two, or three hydrogen atoms by other groups, such as, for example, alkyl groups. Primary amines have the general structures RNH₂ and secondary amines have the general structure R₂NH. The term amine includes, but is not limited to, methylamine, ethylamine, propylamine, isopropylamine, aniline, cyclohexylamine, benzylamine, polycyclic amines, heteroatom substituted aryl and alkylamines, dimethylamine, diethylamine, diisopropylamine, dibutyl amine, methylpropylamine, methyihex ylamine, methylcyclopropylamine, ethylcylohexylamine, methylbenzylamine, methycyclohexylmethylamine, butylcyclohexylamine, morpholine, thiomorpholine, pyrrolidine, piperidine, 2,6-dimethylpiperidine, piperazine, and heteroatom substituted alkyl or aryl secondary amines.

As used herein, the terms “analog” or “derivative” are meant to refer to a chemical compound or molecule made from a parent compound or molecule by one or more chemical reactions. As such, an analog can be a compound with a structure similar to that of EtG or based on an EtG scaffold, but differing from it in respect to certain components or structural makeup, which may have a similar or opposite action metabolically. An analog or derivative of EtG in accordance with the present invention can be used to compete for binding with an antibody that recognizes both the analog and EtG. Also, an analog can include an operative moiety coupled to EtG through a linker group.

As used herein, the term “antibody” is meant to refer to polyclonal and monoclonal antibodies and related antigen recognition units, including fragments and derivatives of immunoglobulin molecules. One method of producing antibodies is to administer an immunogenic derivative of the target analyte, generally combined with an adjuvant such as Freund's adjuvant, in a series of injections to a host animal for the purpose of inducing an immunologic response. Such methods are well known to those skilled in the art. Methods for producing monoclonal antibodies were first described by Kohler and Milstein (Nature, Vol. 256, pp 495-497, 1975; incorporated herein in its entirety) and have been modified several times since the appearance of that publication. For hybridoma technology, the reader is directed generally to U.S. Pat. Nos. 4,491,632; 4,472,500; and 4,444,887; and Methods in Enzymology, 73B:3 (1981); each is incorporated herein in its entirety. Since the particular method is not critical, any proven method can be used to produce an antibody using immunogens as described herein. As used herein, the term “antibody” is meant to refer to a protein that is produced in response to the presence of foreign molecules in the body. They can be characterized by their ability to bind both to antigens and to specialized cells or proteins of the immune system. For example, antibodies are divided into five classes, IgG, IgM, IgA, IgE, and IgD, and are immunoglobulin produced by plasma cells.

As used herein, the term “avidity” is meant to refer to a measure of the overall stability of the complex between antibodies and antigens. The overall stability of an antibody-antigen interaction can be governed by three major factors as follows: (a) the intrinsic affinity of the antibody for the epitope; (b) the valency of the antibody and antigen; and (c) the geometric arrangement of the interacting components. As such, the avidity of the antibody-antigen complex can be modulated by varying the foregoing parameters, as well as others.

As used herein, the terms “carrier,” “immunogenic moiety,” or “immunogenic carrier,” are meant to refer to an immunogenic substance, commonly a protein, which can be coupled to a hapten. An immunogenic moiety coupled to a hapten can induce an immune response and elicit the production of antibodies that can bind specifically with the hapten. Immunogenic moieties are operative moieties that include proteins, polypeptides, glycoproteins, complex polysaccharides, particles, nucleic acids, polynucleotides, and the like that are recognized as foreign and thereby elicit an immunologic response from the host. Examples of polysaccharides are starches, glycogen, cellulose, carbohydrate gums such as gum arabic, agar, and so forth. The polysaccharide can also contain poly(amino acid) residue and/or lipid residues.

In one example, an immunogenic carrier can be coupled with a hapten in order to stimulate immunogenicity and antibody formation against the hapten. Usually, immunogenic carriers are large molecules that are highly immunogenic and capable of imparting immunogenicity to a hapten. For example, a poly(amino acid) immunogenic carrier including, but not limited to, proteins, albumins, serum proteins, ocular lens proteins, and lipoproteins, can be used as an immunogenic carrier because foreign proteins can elicit such an immunological response. Protein carriers can be highly soluble and include functional groups that could facilitate easy conjugation with a hapten molecule. Some of the most common carrier proteins in use today are keyhole limpet hemocyanin (“KLH,” MW 450,000 to 13,000,000), egg ovalbumin, bovine gamma-globulin (“BGG”), and bovine serum albumin (“BSA,” MW 67,000).

As used herein, the term “epitope” is meant to define the region of an antigen that interacts with an antibody. Accordingly, a molecule or other substance, which is an antigen, can include at least one epitope with antibody activity. This can allow for an antigen to have various epitopes recognized by the same or different antibody. Also, an epitope is not an intrinsic property of any particular structure, but can be defined as a binding site that interacts with the antibody.

As used herein, the term “hapten” is meant to refer to a partial or incomplete antigen. They are protein-free substances, mostly low molecular weight substances, that are not capable of stimulating antibody formation, but which do react with antibodies, formed by coupling the hapten to a high molecular weight carrier and then injecting the coupled product, i.e., immunogen, into a human or other animal subject.

As used herein, the term “hetero atom” is meant to refer to an atom other than a carbon atom such as oxygen, nitrogen, sulfur, phosphorus, and the like. Usually, a heteroatom is multivalent so as to form at least two covalent bonds, which can be used in a linking group or other moiety.

As used herein, the terms “immunogen” and “immunogenic” are meant to refer to substances capable of producing or generating an immune response in an organism. An immunogen can also be an antigen. Usually, an immunogen has a fairly high molecular weight (e.g., greater than 10,000), thus, a variety of macromolecules such as proteins, lipoproteins, polysaccharides, some nucleic acids, and certain of the teichoic acids, can be coupled to a hapten in order to form an immunogen in accordance with the present invention.

As used herein, the term “immunogenicity” is meant to refer to the ability of a molecule to induce an immune response, which is determined both by the intrinsic chemical structure of the injected molecule and by whether or not the host animal can recognize the compound. Small changes in the structure of an antigen can greatly alter the immunogenicity of a compound and have been used extensively as a general procedure to increase the chances of raising an antibody, particularly against well-conversed antigens. For example, these modification techniques either alter regions of the immunogen to provide better sites for T-Cell binding or expose new epitopes for B-cell binding.

As used herein, the terms “immunoassay” or “immunodiagnostic” are meant to refer to laboratory techniques that make use of the binding between an antigen and an antibody in order to identify and/or quantify at least one of the specific antigen or specific antibody in a biological sample. Examples of immunoassay can include the following: (1) antibody capture assays; (2) antigen capture assays; (3) two-antibody sandwich assays; and (4) detectable antigen-antibody interactions., Additionally, it is contemplated that new immunoassays will be developed and will be capable of employing the analogs and antibodies of the present invention.

As used herein, the terms “linking group” or “linker” are meant to refer to a portion of a chemical structure that connects two or more substructures such as EtG or an EtG analog, with an operative moiety. A linking group can have at least one uninterrupted chain of atoms other than hydrogen (or other monovalent atoms) extending between the substructures. Usually, a linking group includes a chain of carbon atoms or hetero atoms, which can be substituted or unsubstituted. The atoms of a linking group and the atoms of a chain within a linking group can be interconnected by chemical bonds. For example, linkers maybe straight or branched, substituted or unsubstituted, saturated or unsaturated chains, wherein the chain atoms can include chain or at termini of the chains. Additionally, a linking group may also include cyclic and/or aromatic groups as part of the chain or as a substitution on one of the atoms in the chain. The number of atoms in a linking group or linker is determined by counting the atoms other than hydrogen in the backbone of the chain, which is the shortest route between the substructures being connected. Linking groups may be used to provide an available site on a hapten for conjugating a hapten with an operative moiety such as a tracer, label, carrier, immunogenic moiety, and the like.

As used herein, the terms “label,” “detector molecule,” or “tracer” are meant to refer to any molecule which produces, or can be induced to produce, a detectable signal. The label can be conjugated directly or via a linker to an analyte, immunogen, antibody, or to another molecule such as a receptor or a molecule that binds to a receptor, such as a ligand. Non-limiting examples of labels, detector molecules, or tracers include radioactive isotopes, enzymes, enzyme fragments, enzyme substrates, enzyme inhibitors, coenzymes, catalysts, fluorophores, dyes, chemiluminescers, luminescers, sensitizers, non-magnetic or magnetic particles, solid supports, liposomes, ligands, receptors, hapten radioactive isotopes, and the like. As described herein, the analogs can also be coupled to a variety of labels by methods well known in the art to provide a variety of reagents useful in various immunoassay formats. For detecting the results of the immunoassays, detector molecules such as fluorophores, for example, fluorescein, radio-labels, or chemiluminescent groups can be coupled to the analogs to produce tracers.

As used herein the term “operative moiety” is meant to refer to a molecule or macromolecule coupled to EtG through a linker group. Also, an operative moiety provides an operative function to the EtG for use in preparing or performing immunodiagnostic assays. An operative group can include immunogenic moiety, antigen moiety, tracer moiety, and the like. Usually, an operative group is illustrated as “Z” in the chemical formulas provided below.

As used herein, the terms “sample” or “biological sample” are meant to refer to any quantity of a substance from a living thing, including humans. Such substances include, but are not limited to, blood, serum, urine, tears, cells, organs, tissues, and hair.

As used herein, the term “specificity” is meant to refer to the preferential binding of an antibody with an epitope in comparison with other available epitopes. Also, the specificity of an antibody for binding with EtG can be used to tailor analogs with similar or substantially the same specificity.

Additionally, the terms used herein to describe the invention can be construed using the foregoing definitions and/or definitions well known in the art. As such, the foregoing terminology is meant to describe the invention and is not intended to be limiting.

II. Ethyl Glucuronide Analogs

In one embodiment, the present invention relates to analogs of EtG. As such, EtG can be conjugated with an analog moiety at the 1-carbon or 5-carbon position of the glucyl ring to form an analog. This can include conjugating an analog moiety with the oxygen at the 1-carbon position or with the carbonyl at the 5-carbon position of the glucyl ring. Also, other derivatizations at the 1-carbon or 5-carbon position of the glucyl ring can serve to link EtG with an analog moiety.

An EtG analog can be further coupled through the analog moiety or linker to an operative moiety, such as an immunogenic moiety, antigenic moiety, and/or tracer moiety, to form another analog such as an immunogen, antigen, and/or tracer.

In one embodiment, the present invention describes novel analogs of EtG having conjugations at the 1-carbon position of the glucyl ring. That is, the 1-carbon, oxygen coupled to the 1-carbon, or other derivatization of the 1-carbon position of the glucyl ring can be coupled to a linking moiety. The linker moiety can be considered to be the substituent that is coupled with the EtG scaffold in order to form the analog. The linker moiety can be any of a wide array of chemical entities, which are described in more detail below. Accordingly, the 1-carbon substituted analog of EtG can have the generic structure of Formula 1A and/or Formula 1B:

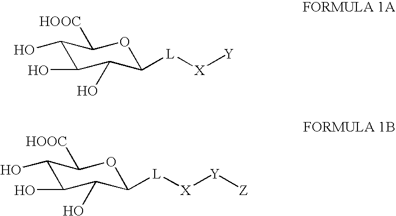

In another embodiment, the EtG scaffold can include a substitution at the 5-carbon position of the glucyl ring. Accordingly, the 5-carbon substitution analog of EtG can have the generic structure of Formula 2A, 2B, 2C, and/or 2D:

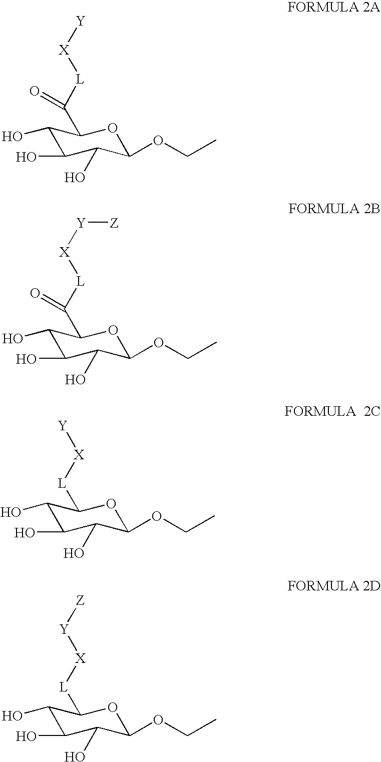

In another embodiment, an operative moiety Z, such as an immunogenic moiety, antigen moiety, label moiety, tracer moiety, and the like, can be conjugated to a plurality of EtG molecules or EtG analogs. This can include the operative moiety being covalently bonded to a plurality of EtG molecules or EtG analogs at the 1-carbon or 5-carbon position of the glucyl ring. Accordingly, the 1-carbon and/or 5-carbon substitution analog of EtG can have the generic structure of Formula 3A, 3B, and/or 3C:

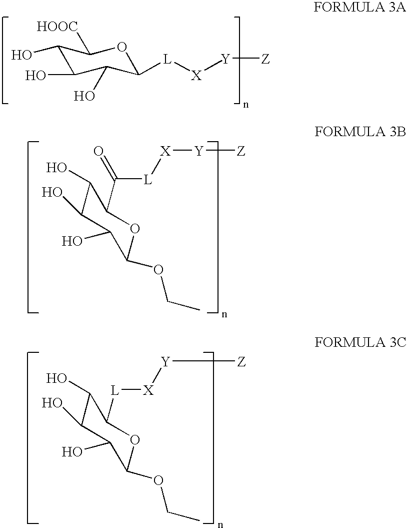

The EtG scaffold depicted in Formulas 1A, 1B, 2A, 2B, 3A, 3B, and/or 3C can be substituted with a wide range of chemical entities. Accordingly, the L group can be an O, S, CO, COO, SO₂, CH₂, (CH₂)ₘ, NH, NH(CH₂)₂NH, CONH, Ph, NHCH₂Ph, and the like. As such, the L group can be used as a linking group to conjugate the analog moiety and/or conjugate moiety to the EtG scaffold. With respect to Formulas 3A, 3B and/or 3C, n can be greater than or equal to about 1, more preferably less than about 1000, and most preferably less than about 500 to 100, which varies with the protein or other carrier. Additionally, m can be greater than or equal to 1, and is usually less than 20, preferably less than 10.

Additionally, as used in connection to Formulas 1A, 1B, 2A, 2B, 3A, 3B, and/or 3C, the X group can be comprised of an aliphatic group, aromatic group, as well as a saturated or unsaturated, substituted or unsubstituted, and/or straight or branched chain having 1-20 carbon or hetero atoms, or more preferably 1-10 carbon or hetero atoms. Some examples of substitution groups include primary and secondary amines, aliphatics, carbonyl groups, halogens, and the like. Also, the X group can include a cyclic group that is substituted or unsubstituted, or a substituted or unsubstituted aromatic or aliphatic group having 1-2 rings, polycyclic aromatic rings, hetero aromatic rings, and the like. The X group can also be a substituted or unsubstituted aliphatic linking group containing 1-20 or 1-10 chain atoms of carbon or hetero atoms in place of or in addition to a ring group. Furthermore, the X group can be any type of bond between L and Y. Also, X can be any combination of the foregoing groups.

The Y group can be an end group or coupling group, which can be used for coupling the linker group with an operative moiety, such as a carrier, label, immunogenic moiety, and the like. In some instances, the end group can be derivatized or coupled with a carrier, tracer moiety, or immunogenic moiety via chemical syntheses well known in the art, wherein the Y group can be a reactive group that is used to couple with the Z group. As such, Y can be various groups, such as aliphatics, alcohols, amines, amides, carboxylic acids, aldehydes, esters, activated esters, aliphatic esters, imidoesters, isocyanates, isothiocyanates, anhydrides, thiols, alcohols, thiolactones, diazonium groups, maleimido groups, and the like as well as groups derived therefrom. Also, Y can be a Y₁—Z group, wherein Y₁ is derived from the Y end group being coupled to the Z group.

Furthermore, the Z group can be nothing or any operative moiety that can be coupled to the linker moiety. As such, the L—X—Y group can be considered to be the analog moiety and the Z group can be an operative group. The linker moiety can functionally serve as a linker or linking group between the EtG scaffold and an operative moiety. For example, the operative moiety can be a carrier, label, tracer, protein, enzyme, enzyme fragment, fluorescent compound, phosphorescent compound, thermochromic compound, photochromic compound, anti-stokes up-regulating compound, chemiluminescent material, electrochemical mediator,-particle, reporter group, enzyme inhibitor, nucleic acid, polypeptide, and the like.

For example, in each of Formulas 1A, 1B, 2A, 2B, 3A, 3B, and/or 3C the Y group can comprise an end group or linker derived from the end group. Illustratively, Y can be any of the following end groups or a linker group derived therefrom: COOH (carboxylic acid); COO; COO—NHS (NHS active ester); NHS; COO-tertbutyl; OH; SH; —O—NHS (NHS active ester linker); COOCH₂CH₃; COOCH₃; OCH₂CH₃; OCH₃; NH; NH₂; NHCO (amide); combinations thereof; and the like. More preferably, when Y is end group, it can be selected from the group consisting of NHS, COOH, COO—NHS, COO-tertbutyl, OH, O—NHS, COOCH₂CH₃, COOCH₃, OCH₂CH₃, OCH₃, or NH₂. On the other hand, when Y is a linker, it is Y₁—Z, wherein Y₁ can be preferably selected from the group consisting of is at least one of COO, CO, O, CONH, or NH and Z is a macromolecule.

Accordingly, the Z group or operative moiety can be a carrier, tracer, or a label, such as a protein, enzyme, enzyme fragment, fluorescent compound, chemiluminescent material, electrochemical mediator, particle, reporter group, enzyme inhibitor, and/or nucleic acid. Illustratively, Z can be any of the following macromolecule groups: (a) BSA; (b) KLH; (c) fluorescent tracer; (d) chemiluminescent group; (e) enzyme (e.g., G6PDH); (f) enzyme donor; (g) enzyme acceptor; and (h) like carriers, tracers, or labels.

Generally, the analogs can include a variety of operative moieties by methods well known in the art to provide a variety of reagents useful in various immunoassay formats. As such, detector molecules, such as fluorophores, radio-labeled, or chemiluminescent groups, can be used to produce tracers. The analogs can also be bound to microparticles, such as colored latex, for use in spectrophotometric or direct optical detection formats such as in latex agglutination and chromatographic strip tests. The operative moiety may also be an indirect detection molecule, such as an energy transfer partner, enzyme or other group, which is detected by further chemical reactions.

Accordingly, coupling an operative moiety with the analog can be accomplished by any chemical reaction that will couple the operative moiety. This linkage or coupling can include many chemical mechanisms, for instance covalent binding, affinity binding, intercalation, coordinate binding, and complexing. Most often, the linkage or coupling is made through covalent bonding. Covalent binding can be achieved either by direct condensation of existing side chains or by incorporation of external bridging molecules. Many bivalent or polyvalent linking agents can be useful in coupling protein molecules, such as a carrier, to the analog. Representative coupling agents that can be used as or in addition to Y can include organic compounds such as thioesters, carbodiimides, N-hydroxysuccinimide esters, diisocyanates, glutaraldehyde, diazobenzenes, and hexamethylene diamines; however, this listing is not an exhaustive compilation of the various classes of coupling agents known in the art but, rather, is representative of the more common coupling agents.

III. Ethyl Glucuronide Immunogens

Implementing an immunoassay for the detection of a small molecule, such as EtG, can be a challenge. This is because such small molecules can often lack antigenicity, making it difficult to generate antibodies. It is particularly problematic with EtG, which lacks immunogenicity. To increase the immunogenicity, larger antigenic compounds including, but not limited to, BSA, ovalbumin, KLH, and the like, can be coupled to EtG. Further, detection of EtG in an immunoassay generally requires the use of a detectable tracer conjugated to an antibody, EtG, or EtG analog.

Accordingly, coupling an immunogenic operative moiety to EtG can provide an EtG immunogen that is sufficiently immunologically similar to EtG so that antibodies induced by the immunogen can react with the immunogen, EtG, and other EtG analogs. As such, an immunogen based on EtG is also considered an EtG analog. EtG analogs in accordance with the present invention which include an immunogenic carrier can be capable of inducing the production of anti-EtG antibodies, such as monoclonal and polyclonal antibodies. Accordingly, the antibodies generated using unique EtG immunogens can interact and/or bind with EtG and other EtG analogs. These antibodies, immunogens, antigens, and analogs can be useful in preparing for and performing immunoassays for the detection of EtG in biological samples.

Immunogens can be made by coupling EtG to an immunogenic or antigenic carrier protein through a linker at either the 1-carbon or 5-carbon position of the glucyl ring of EtG or an EtG analog, as shown in Formulas 1B, 2B, 3A, 3B, and/or 3C. Also, it has been found in some instances that longer linkers can increase the affinity of the antibodies produced. In part, it is thought, without being bound thereto, that longer linkers can allow more accessibility to the antigen. Also, due to the increased surface area of the exposed antigen or epitope, the avidity may also be increased, providing an improvement in the art.

An immunogenic moiety can include various proteins or polypeptides, which can function as an immunogenic carrier. These types of polypeptides include albumins, serum proteins, globulins, ocular lens proteins, lipoproteins, and portions thereof. Illustrative proteins include BSA, KLH, egg ovalbumin, bovine gamma-globulin (“BGG”), and the like. Alternatively, synthetic polypeptides may be utilized. Additionally, an immunogenic moiety can also be a polysaccharide, which is a high molecular weight polymer. Examples of polysaccharides are starches, glycogen, cellulose, carbohydrate gums such as gum arabic, agar, and the like. Also, an immunogenic moiety can be a polynucleotide, such as DNA or RNA. The polynucleotide can be modified or unmodified, and comprised of any number of nucleic acids so long as it provides the carrier and/or immunogenic functionality. The polysaccharide can also contain or link to a polypeptide residue, polynucleotide residue, and/or lipid residue. Furthermore, an immunogenic moiety can either be a polynucleotide alone or conjugate to one of the polypeptides or polysaccharides mentioned above.

An immunogenic moiety or carrier can also be a particle or microparticle. The immunogenic particles are generally at least about 0.02 microns (μm) and not more than about 100 μm, and usually about 0.05 μm to 10 μm in diameter. The particle can be organic or inorganic, swellable or non-swellable, and/or porous or non-porous. Optionally, an immunogenic particle can have a density approximating water, generally from about 0.5 to 1.5 g/ml, and be composed of a material that can be transparent, partially transparent, or opaque. The immunogenic particles can be biological materials such as cells and microorganisms, including non-limiting examples such as erythrocytes, leukocytes, lymphocytes, Streptococcus, Staphylococcus aureus, E. coli, and viral particles. The particles can also be comprised of organic and inorganic polymers, liposomes, latex, phospholipid vesicles, liposomes, cationic liposomes, anionic liposomes, lipoproteins, lipopolymers, and the like.

In one embodiment, the present invention relates to immunogens prepared from the forgoing EtG analogs. Namely, the analogs of Formulas 1B, 2B, 3A, 3B and 3C can include the linker moieties as described above, and Z can be an immunogen. As such, Z can be any immunogenic moiety that can elicit an immunological response and provide for antibodies to be produced that target at least a portion of the EtG analog. Also, with reference to Formulas 3A, 3B, and 3C, n can be greater than or equal to 1, preferably from about 1 to about 1000, more preferably from about 1 to about 500, and most preferably from about 1 to about 100. For example, when Z is a particle, n can be greater than 1000. When Z is BSA, n can be from about 1 to about 35, and when Z is KLH, n can be from about 1 to about 500.

Thus, the immunogens prepared in accordance with the present invention can be used to generate antibodies that can have an affinity for EtG as well as EtG analogs.

IV. Anti-EtG Antibodies

In one embodiment, an EtG analog-based immunogen in accordance with the present invention can be used in an embodiment of a method for producing monoclonal and/or polyclonal antibodies. As such, antibodies can be produced from the EtG-based immunogen and interact and/or bind with EtG. This can allow for the analogs of the present invention to be useful in preparing antibodies for use in immunoassays for identifying the presence of EtG. Also, methods of producing antibodies with immunogens are well known in the art. The immunogens can be used in the screening for the monoclonal and/or polyclonal antibodies that interact and/or bind with EtG.

For example, a well-known method for obtaining antibodies can be utilized with an EtG-based immunogen in order to prepare anti-EtG antibodies. As such, an immunogen based on an EtG analog can be obtained and combined with an immunogenic formulation. Briefly, about 0.5 mL of an immunogen composition is admixed with about 0.5 mL of complete Freund's adjuvant; however, other amounts of immunogen and/or adjuvant can be used. The immunogenic formulation can then be administered to an antibody producing subject, which can be a rat, mouse, pig, rabbit, bird, sheep, and/or other animal, but preferably a mammal. The administration can be via tail vein injection, subcutaneous injection, intravenous injection, or other well-known injection sites. Subsequently, immunogenic boosters can be administered to the animal that received the initial administration, wherein the booster can include substantially the same ingredients as the initial formulation and can be administered at predetermined intervals. For example, the initial administration can be followed by subsequent boosters once a week or at other longer or shorter intervals. After at least the initial administration, and optionally after subsequent boosters, the anti-EtG antibodies produced by the animal can be collected. The antibodies can be collected by obtaining blood, serum, plasma, or other biological sample from the animal previously administered the immunogen. Optionally, the antibody-containing composition can then be processed as is well known in the art, wherein such processing can include techniques that place the antibodies into a format suitable for performing an immunodiagnostic assay. Alternatively, the processing can include screening the antibodies with ELISA by well-known and established techniques. Additionally, the processing can be used to obtain polyclonal antibodies as is well known in the art.

V. Immunodiagnostic Assays

The anti-EtG antibodies, either monoclonal or polyclonal, can be used in immunoassays for identifying the presence of EtG in a sample, such as blood, plasma, serum, tissue, and the like. This can be beneficial for identifying or determining whether or not a subject has ingested alcoholic beverages. Thus, the anti-EtG antibodies can be used in immunodiagnostic assays in place of other antibodies so that the assays can be configured for identifying the presence and optionally quantifying the amount of EtG, which is an indication of alcohol consumption. Additionally, the immunodiagnostic assays can use EtG analogs in accordance with the present invention or other EtG analogs.

A. Fluorescence Polarization Immunoassay

Fluorescence polarization immunoassay (“FPIA”) technology is based upon competitive binding between an antigen/drug in a sample and a known concentration of labeled antigen/drug. FPIA technology is described in U.S. Pat. Nos. 4,593,089; 4,492,762; 4,668,640; and 4,751,190, which are incorporated herein by reference. Accordingly, the FPIA reagents, systems, and equipment described in the incorporated references and those well known in the art can be used with anti-EtG antibodies which are also anti-EtG analog antibodies.

The FPIA technology can be used to identify the presence of EtG and can be used in assays that quantify the amount of EtG in a sample. In part, the rotational properties of molecules in solution allow for the degree of polarization to be directly proportional to the size of the molecule. Accordingly, polarization increases as molecular size increases. That is, when linearly polarized light is used to excite a fluorescent-labeled or other luminescent-labeled EtG or analog thereof, which is small and rotates rapidly in solution, the emitted light is significantly depolarized. When the fluorescent-labeled EtG or analog interacts with or is bound to an antibody, the rotation is slowed and the emitted light is highly polarized. This is because the antibody significantly and measurably increases the size of the complex. Also, increasing the amount of unlabeled EtG in the sample can result in decreased binding of the fluorescent-labeled EtG or analog by the anti-EtG antibody, and thereby decrease the polarization of light emitted from sample. The quantitative relationship between polarization and concentration of the unlabeled EtG in the sample can be established by measuring the polarization values of calibrations with known concentrations of EtG. Thus, FPIA can be used to identify the presence and concentration of EtG in a sample.

B. Homogeneous Microparticle Immunoassay

Homogeneous microparticles immunoassay (“HMI”) technology, which can be referred to as an immunoturbidimetric assay, is based on the agglutination of particles and compounds in solution. When particles and/or chemical compounds agglutinate, particle sizes can increase and, thereby, increase the turbidity of a solution. Accordingly, anti-EtG antibodies can be used with microparticles and EtG analogs in order to assess the presence, and optionally the amount, of EtG in a sample. HMI technologies can be advantageous because the immunoassays can be performed on blood, blood hemolysate, serum, plasma, tissue, and/or other samples. HMI assays can be configured to be performed with EtG and/or an analog loaded onto a microparticle, or with an anti-EtG antibody loaded onto a microparticle. The use of an EtG analog loaded microparticle can be especially advantageous because of the ability to efficiently load the microparticle. In any event, HMI or immunoturbidimetric assays are well known in the art for measuring agglutination of substances in a sample.

C. Cloned Enzyme Donor Immunoassays

Cloned enzyme donor Immunoassays (“CEDIA®” a trademark of Roche Diagnostics) has proven to be a highly accurate and effective method for identifying the presence and determining the amount of small molecules in samples. CEDIA® is based upon the re-association and/or re-activation of enzymatically inactive polypeptide fragments of β-galactosidase. CEDIA® hats proven to be a highly accurate method for quantitative measurements of therapeutic drugs and drugs of abuse. CEDIA® is the subject of several patents as follows: U.S. Pat. No. 4,708,929, which claims competitive homogeneous assay methods; U.S. Pat. No. 5,120,653, which claims a recombinant DNA sequence for coding the enzyme donor fragment and a host for such a vector; U.S. Pat. No. 5,604,091, which claims amino acid sequences of the enzyme donor fragment; and U.S. Pat. No. 5,643,734, which teaches and claims kits for CEDIA® assays, wherein all of the foregoing patents are incorporated herein by reference. In particular, a β-galactosidase enzyme donor polypeptide fragment can combine with a β-galactosidase enzyme acceptor fragment in order to form an active β-galactosidase enzyme. The active enzyme complex can be capable of transforming a substrate into a product that is differentially detectable. Usually, the product is a different color from the substrate and is quantified using spectrophotometric methods.

Additionally, the CEDIA® technology can be compatible with various small molecule-based antigens and immunogens. As such, conjugating a hapten or other small analyte or analyte analog to the enzyme donor fragment at certain sites does not substantially affect the ability to form the active enzyme by the complementation reaction, and does not substantially affect the rate of enzymatic activity when in the presence of a substrate for β-galactosidase. However, when the enzyme donor-hapten conjugate is bound by an anti-analyte antibody, for example, when little or no analyte is present in a sample being tested, the complementation reaction is inhibited, thereby reducing the amount of active enzyme present in the reaction mixture. Hence, the enzyme-catalyzed reaction rate can be decreased under such conditions. In contrast, when the sample that is tested contains significant concentrations of a target analyte, the analyte competes with the enzyme donor-hapten for binding sites on the anti-analyte antibody, thereby increasing the amount of active enzyme formed by the complementation reaction. Therefore, the enzyme-catalyzed reaction rate is directly proportional to the concentration of target analyte present in the specimen tested.

As such, an embodiment of the present invention uses CEDIA® technology so that the competition of EtG in the biological sample with an EtG-based analog coupled to an inactive genetically engineered enzyme-donor (“ED”) fragment can provide an indication as to the presence and/or amount of EtG in a sample. The ED fragment can typically be derived from β-D-galactoside galactohydrolase or β-galactosidase (“β gal”) from E.coli, or others. In the instance EtG is present in the sample, it binds to the antibody, leaving the ED portion of the ED-analog conjugate free to restore enzyme activity of β-D-galactoside galactohydrolase or β gal in the reaction mixture so as to be capable of association with enzyme acceptor (“EA”) fragments. The active enzyme comprised of the ED and EA is then capable of producing a quantifiable reaction product when exposed to an appropriate substrate. A preferred substrate is chlorophenol red-β-D-galactopyranoside (“CPRG”), which can be cleaved by the active enzyme into galactose and CPR, wherein CPR is measured by absorbency at about wavelength 570 nm. In the instance EtG is not present in the sample, the antibody binds to the ED-analog conjugate, thereby inhibiting association of the ED fragments with the EA fragments and inhibiting restoration of enzyme activity. The amount of reaction product and resultant absorbance change are proportional to the amount of EtG in the sample.

For example, a method for performing a CEDIA® assay can be used in accordance with the present invention. Accordingly, an EtG-ED conjugate and a corresponding EA can be obtained by methods in accordance with the present invention. Additionally, a sample, such as a biological sample suspected of containing EtG can be obtained. Anti-EtG antibody, which can also interact with the EtG-ED conjugate can be obtained by methods in accordance with the present invention. Known amounts or concentrations of the EtG-ED conjugate, EA, and anti-EtG antibody can be obtained and formulated into separate compositions, such as a standard buffer system, for use in a competitive binding assay. The EtG-ED conjugate and anti-EtG antibody are then combined with the biological sample into a reaction solution. Optionally, the EA is also combined into the reaction solution at this point or after a sufficient time for competitive interactions with the antibody have been able to occur. A competitive reaction takes place between the known amount of EtG-ED conjugate and EtG in the biological sample with the known amount of anti-EtG antibody in the reaction solution. After the competitive reactions and the EA has been introduced into the reaction solution, an ED-EA enzyme-cleavable substrate can be introduced into the reaction solution. The enzyme activity between the ED-EA enzyme and enzyme-cleavable substrate can be measured by measuring the absorbance of a cleavage product or other well-known measuring technique. The measurement of enzyme activity can be used to determine whether or not EtG is present in the sample. Additionally, comparing the measurements obtained from the biological sample with standardized measurements obtained from known concentration standards can be used to quantify the amount of EtG in the sample, and thereby identify the amount of EtG in a person suspected of consuming alcohol.

Preferred substrates for use in immunoassays utilizing-β-galactosidase include those described in U.S. Pat. Nos. 5,032,503; 5,254,677; 5,444,161; and 5,514,560, which are incorporated herein by reference. Chlorophenol-red-β-D-galactopyranoside is an exemplary substrate.

One embodiment of the present invention is a CEDIA® assay system for detecting EtG. An example of components of the CEDIA® system can include the following: i) monoclonal or polyclonal anti-EtG antibodies capable of binding to EtG, EtG analog, and/or EtG-ED or EtG-EA; ii) a sample suspected of containing the EtG from alcohol consumption; iii) EtG analog coupled to an ED or EA; and/or iv) one of an ED or EA that will associate with the EtG-ED or EtG-EA for restoring enzymatic activity so that an ED and EA are present in the system. Alternatively, the assay system can be provided as a kit exclusive of the sample. Additionally, the assay system can include various buffer compositions, EtG concentration gradient compositions or a stock composition of EtG, and the like.

D. Enzyme Multiplied Immunoassay Technique

A competitive assay using an enzyme multiplied immunoassay technique (“EMIT”) can also be used to assess whether or not EtG is present in a sample. EMIT is based upon the ability of an enzyme bound to analyte, such as EtG or EtG analog, to have enzymatic activity in the presence of an anti-analyte antibody. In the instance a sample is devoid of the free analyte, the anti-analyte antibody interacts with the enzyme-bound analyte and renders the enzyme incapable of processing the enzyme substrate. On the other hand, in the instance a sample includes the free analyte, the anti-analyte antibody interacts with the free analyte so that the enzyme that is bound to the analyte retains the capability of processing the enzyme substrate. As such, increasing the amount of analyte in a sample increases the overall enzymatic activity of the enzyme bound to the analyte, and decreasing the amount of analye in the sample decreases the overall enzymatic activity of the enzyme bound to the analyte. EMIT has proven to be a highly accurate method for quantitative measurements of therapeutic drugs and drugs of abuse. Additional information regarding the EMIT technology can be reviewed in U.S. Pat. No. 3,817,837, which is incorporated herein by reference.

Additionally, the EMIT technology can be compatible with various small molecule-based antigens and immunogens, such as EtG antigens and EtG immunogens. As such, conjugating an EtG analog to the enzyme at certain sites does not substantially affect the ability or the rate of enzymatic activity when in the presence of a substrate for the enzyme. However, when the enzyme-EtG conjugate is bound by an anti-EtG antibody, for example, when little or no EtG is present in a sample being tested, the enzymatic reaction is inhibited, thereby reducing the amount of active enzyme present in the reaction mixture. Hence, the enzyme-catalyzed reaction rate can be decreased under such conditions. In contrast, when the sample that is tested contains significant concentrations of EtG, the EtG competes with the enzyme-EtG for binding sites on the anti-EtG antibody, thereby increasing the amount of active enzyme. Therefore, the enzyme-catalyzed reaction rate is directly proportional to the concentration of EtG present in the specimen tested.

E. Chemiluminescent Heterogeneous Immunoassays

A competitive assay using chemiluminescent microparticle immunoassay (“CMIA”) technology can also be used to assess whether or not EtG is present in a sample. Various types of CMIA technologies are well known in the art of heterogeneous immunoassays for determining the presence and/or amount of a chemical entity in a sample. Some CMIA technologies can be exemplified by U.S. Pat. Nos. 6,448,091; 5,798,083; and 5,834,206, which are incorporated herein by reference. CMIA assays can include the use of anti-EtG antibodies, which are capable of binding to EtG and its analogs, which are coupled to particles, such as magnetic particles or particles suitable for separation by filtration, sedimentation, and/or other means. Additionally, a tracer, which can include an EtG analog linked to a suitable chemiluminescent moiety, can be used to compete with free EtG in the patient's sample for the limited amount of anti-EtG antibody on the particle. After the sample, tracer, and antibody particles interact and a routine wash step has removed unbound tracer, the amount of tracer bound to antibody particles can be measured by chemiluminescence, wherein chemiluminescence is expressed in Relative Light Units (RLU). The amount of chemiluminescence is inversely related to the amount of EtG in the patient's sample and concentration is determined by constructing a standard curve using known concentrations.

F. Other Immunoassays

The EtG analogs, conjugates, antibodies, immunogens and/or other conjugates described herein are also suitable for any of a number of heterogeneous immunoassays with a range of detection systems including but not limited to enzymatic or fluorescent, and/or homogeneous immunoassays including but not limited to rapid lateral flow assays, and antibody arrays, as well as formats yet to be developed.

While various immunodiagnostic assays have been described herein that utilize the EtG analogs, conjugates, antibodies, immunogens and/or tracers, such assays can also be modified as is well known in the art. As such, various modifications of steps or acts for performing such immunoassays can be made within the scope of the present invention.

### EXAMPLES

The following examples are provided to illustrate embodiments of the invention and are not intended to be limiting. Accordingly, some of the examples have been performed via experiment and some are prophetic based on techniques, standards, and results well known in the art. Also, it should be apparent that the invention can include additional embodiments not illustrated by example. Additionally, many of the examples have been performed with experimental protocols well known in the art using the EtG analogs, antigens, immunogens, and anti-EtG antibodies prepared in accordance with the present invention.

### Example 1

### Preparation of an Immunogenic EtG Analog

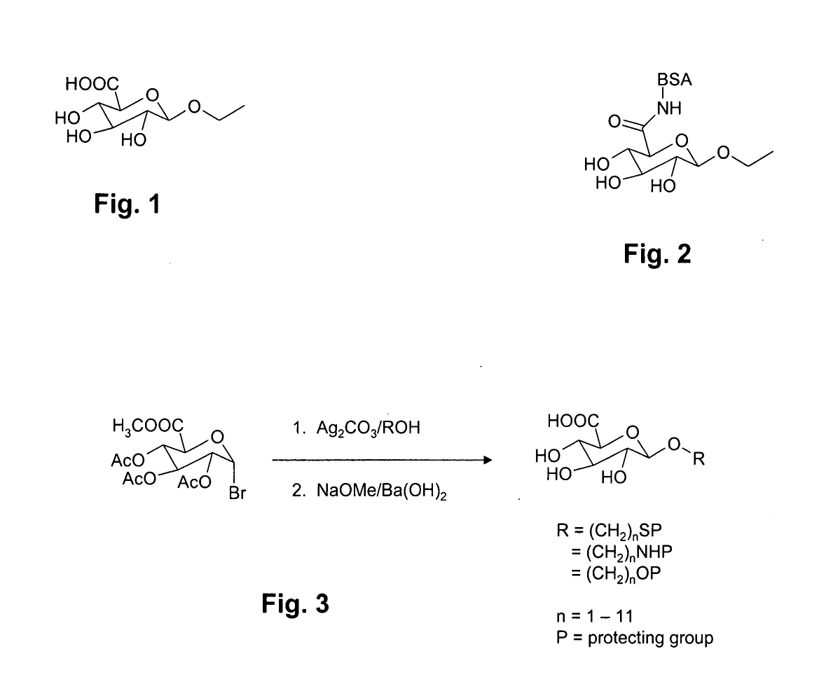

The structure of EtG is shown in FIG. 1. In general, coupling of EtG to an immunogenic carrier can be accomplished by any number of chemical reactions known in the art. Preferably, a linker is used and typically the linker is covalently bound to the hapten (e.g., EtG) and/or the carrier. Covalent binding can be achieved either by direct condensation of existing side chains or by incorporation of external bridging molecules. Many bivalent or polyvalent linking agents are useful in coupling protein molecules, such as a carrier, to other molecules. Representative linkers include organic compounds such as thioesters, carbodiimides, N-hydroxysuccinimide esters, diisocyanates, glutaraldehyde, diazobenzenes, and hexamethylene diamines, as well as those listed above and others well known in the art.

An exemplary method for preparing an immunogenic EtG derivative is now described; however, it should be recognized that other methods may be employed within the scope of the present invention. A reaction mixture is prepared from a stirred solution of ethyl glucuronide (6.7 mg, 0.03 mmol) in 1 ml of dimethyl formamide (DMF) to which 1-ethyl-3-(3-dimethylaminopropyl)carbodiimide hydrochloride (EDAC) (14.4 mg, 0.075 mmol) and N-hydroxy-5-norbornene-2,3-dicarboximide (NHDC) (13.5 mg, 0.075 mmol) are added. The reaction mixture is stirred at room temperature overnight.

The resulting EtG-NHDC ester is reacted with an immunogenic carrier including, but not limited to, BSA, KLH, ocular lens protein, ovalbumin, or any immunogenic fragment of an appropriate carrier. For example, a solution of BSA (40 mg) in 0.1 M phosphate buffered saline (PBS) at pH 7.0 and DMF (2 ml PBS, 0.1 ml DMF) can be combined with 1 ml of the EtG-NHDC ester solution described above. The reaction mixture is stirred at room temperature for approximately 5 hours, and then purified by dialysis against PBS at pH 7.0, using about 3 changes of approximately 2 liters each, for a minimum of about 7 hours to yield approximately 40 mg of EtG-BSA immunogen. Referring to FIG. 2, the immunogenic carrier is attached via a linker attached to the carboxyl moiety at the 5-carbon position of the glucyl ring of EtG when prepared by this method.

Alternatively, other glucuronide derivatives are useful for preparing an immunogen or other conjugates. In particular, as shown in FIG. 3, a mercapto, or amino alkoxy moiety, or other alcohol replacing the ethanol group at the 1-carbon position of the glucyl ring can provide a suitable derivative for coupling to an immunogenic carrier, label, solid substrate, or the like. For example, triacetyl-α-D-6-bromoglucosiduronic acid methyl ester (“BGAME”) (1 g) can be dissolved in absolute benzene (20 ml) in the absence of light, before 5 ml of 52.8 mM tert-butanol (or other alcohols can be used) is used. The reaction is heated to boiling and within about 3 hours, silver carbonate (1.5 g) is added. The reaction mixture is stirred overnight at room temperature. The resulting bromide precipitate is harvested via centrifugation and decanting of the solvent phase. Remaining solvent is removed under vacuum. The yellowish residue contains colorless crystals of tert-butylglucosiduronic acid methyl ester (“t-BuGAME”), approximately 0.34 g.

Absolute methanol (5 ml) is used to dissolve t-BuGAME (0.34 g, 0.87 mM), and sodium methanolate (1 ml of 1% in absolute methanol) is added. The reaction mixture is stirred overnight at room temperature. After evaporation of the solvent, the yellow residue is suspended in barium hydroxide solution (5 ml of 0.03% aqueous solution) for approximately 1 hour. The pH of the suspension is adjusted to about pH 6.0 by drop-wise addition of oxalic acid (9% solution) and the precipitate is removed by centrifugation and evaporated under vacuum until dry. The residue is solubilized in several drops of methanol with shaking, and upon drop-wise addition of diethyl ether, a white precipitate forms, representing approximately 0.2 g of tert-butyl glucuronide. As mentioned above, other alcohols may be substituted for tert-butanol to form the glucuronide derivative, particularly those containing mercapto or amino alkoxy moieties. Such derivatives are then coupled to immunogenic carriers at the R group by standard methods known in the art.

### Example 2

### Preparation of Anti-EtG Antibodies

As discussed above, any number of established procedures may be used to prepare antibodies using an immunogen as described above. For example, the immunogen shown in FIG. 2, in Freund's adjuvant, was used to immunize mice. Following a series of immunizations, as routine in the art, the spleens were removed and fused with an immortal non-producing myeloma cell line to produce hybridoma cell lines using methods known in the art. For the purposes of the present invention, various hybridoma cell lines (e.g., 19D7, 14C5, and 12E7 clones) were selected for production of monoclonal antibodies, after preliminary screening of culture supernatant for capacity to recognize EtG. Although several hybridoma cell lines showed positive reactivity when screened against EtG, hybridoma cell lines 19D7, 14C5, and 12E7 clones, were selected for a variety of reasons, including its growth and antibody production characteristics.

### Example 3

### Preparation of EtG-MPA Analog

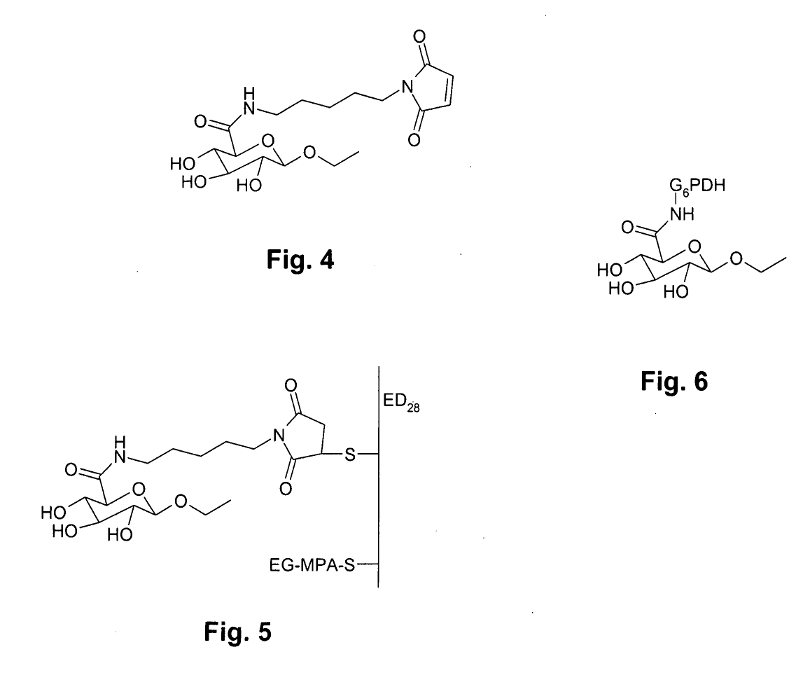

Referring to FIG. 4, a preferred hapten-linker complex for coupling to an enzyme or enzyme fragment is a maleimide adduct or analog form of the hapten. As an example, a solution of EtG (10 mg, 0.0457 mM) in DMF (1 ml) is combined with EDAC (22 mg, 0.113 mM) and N-hydroxysuccinimde (“NHS”) (13 mg, 0.113 mM) to form a reaction mixture. The reaction mixture is stirred at room temperature for approximately 3 hours. Maleimidopentylamine (“MPA”) hydrochloride (31 mg, 0.135 mM) is added to the reaction mixture and the pH of the mixture is adjusted to pH 8.0 with drop-wise addition of triethyl amine. The solution is stirred at room temperature for approximately 30 minutes and then purified by HPLC to yield approximately 10 mg of EtG-MPA as a white solid.

### Example 4

### Preparation of EtG-MPA-ED28 Conjugate

A preferred β-galactosidase enzyme donor for use in connection with CEDIA is ED28, a polypeptide containing residues 6-45 of β-galactosidase, with cysteines at positions 1 and 46 (relative to the numbering of the original β-galactosidase fragment). Typical linker groups used are maleimide adducts as described above. For example, to a solution of ED28 enzyme (1 mg) in PBS (0.714 ml at pH 7) is added a solution of 0.24 mg EtG-MPA in 0.3 ml of DMF. The solution is mixed by vortexing for 10 seconds and maintained at room temperature for approximately 2 hours. The resulting ED28-EtG-MPA conjugate, as shown in FIG. 5, is purified by HPLC. Essentially, each ED28 polypeptide couples with up to two EtG-MPA complexes.

### Example 5

### Preparation of EtG-G6PDH Conjugate

In an exemplary method, EtG (10 mg) is reacted with NHS (6.22 mg) and EDAC (9.51 mg) in DMF solvent (1 ml) with stirring overnight at room temperature. The resulting activated ester is added to a solution of glucose-6 phosphate dehydrogenase (“G6PDH”) (15.9 mg), glucose-6-phosphate (G6P) (119.2 mg) and DMF (0.7 ml) in Tris buffer (2 ml) at pH 8.0 at ice bath temperature. The reaction is monitored, preferably using an anti-EtG antibody as described herein, to inhibit G6PDH activity as determined spectrophotometrically at 340 nm by measuring the enzyme's capacity to convert nicotinamide adenin dinucleotide (“NAD”) to NADH. When the enzymatic activity of G6PDH shows 60-75% inhibition in the presence of the anti-EtG antibody relative to its enzymatic activity in the absence of the antibody, the reaction is stopped and EtG-G6PDH conjugate, as shown in FIG. 6, is purified by chromatographic separation on G25 Sephadex using 50 mM Tris-HCl at pH 8.0 with sodium azide as a preservative.

### Example 6

### Homogeneous Competitive Immunoassays

G6PDH-EtG conjugate is used to compete with EtG in urine, serum, or other samples for binding to an anti-EtG antibody, for example, a monoclonal antibody made according to procedures described herein. In the absence of EtG in the sample tested, the anti-EtG antibody binds to the G6PDH-EtG conjugate, inhibiting enzymatic activity. When EtG is present in a sample, it competes for binding sites on the anti-EtG antibodies, leaving at least some of the EtG-G6PDH unbound and capable of reacting with the substrate. Thus, as the concentration of EtG in a sample increases, the amount of enzymatic activity increases proportionately.

Exemplary reagents for performing such an assay include: anti-EtG antibody, which is preferably a monoclonal antibody with sensitivity and specificity similar to that produced by hybridoma cell lines 19D7, 14C5, or 12E7; substrate reagent comprising 8.5 mM glucose-6-phosphate and 5.25 mM NAD in Tris buffer at pH 5.0, which can be combined with sodium azide as a preservative; enzyme conjugate reagent comprising EtG-G6PDH conjugate as described herein in Tris buffer at pH 8.0, which can be combined with sodium azide as a preservative; and calibrators, for example, different concentrations of EtG (such as 0, 0.2, 0.5, 1.0, 2.5, 5.0 mg/dL) in a buffer or urine-buffer solution or other buffered solution suitable for use with the particular sample type to be tested, at pH 6.0. Preferably, the anti-EtG antibody is mixed into the substrate reagent. The above reagents may be packaged together as a kit with or without the calibrators, which may be packaged separately.

A calibration curve is established using, for example, Hitachi analyzer (917 or 717 or Olympus AU 640), or other comparable instrument. To use the Hitachi 917 analyzer, 35 microliters of calibrator is mixed with 80 microliters of substrate reagent containing anti-EtG antibodies and 80 microliters of enzyme conjugate reagent. To use the Hitachi 717 instrument, 20 microliters of calibrator is added to 125 microliters of substrate reagent containing anti-EtG antibodies and 125 microliters of enzyme conjugate reagent, and the instrument is used according to manufacturer's recommendations. To use the Olympus AU640 instrument, 50 microliters of calibrator is added to 80 microliters of substrate reagent containing anti-EtG antibodies and 80 microliters of enzyme conjugate reagent, and the instrument is used according to manufacturer's recommendations. When testing specimens (urine, serum, etc.), a sample of the specimen is substituted for the calibrator in the above procedures.

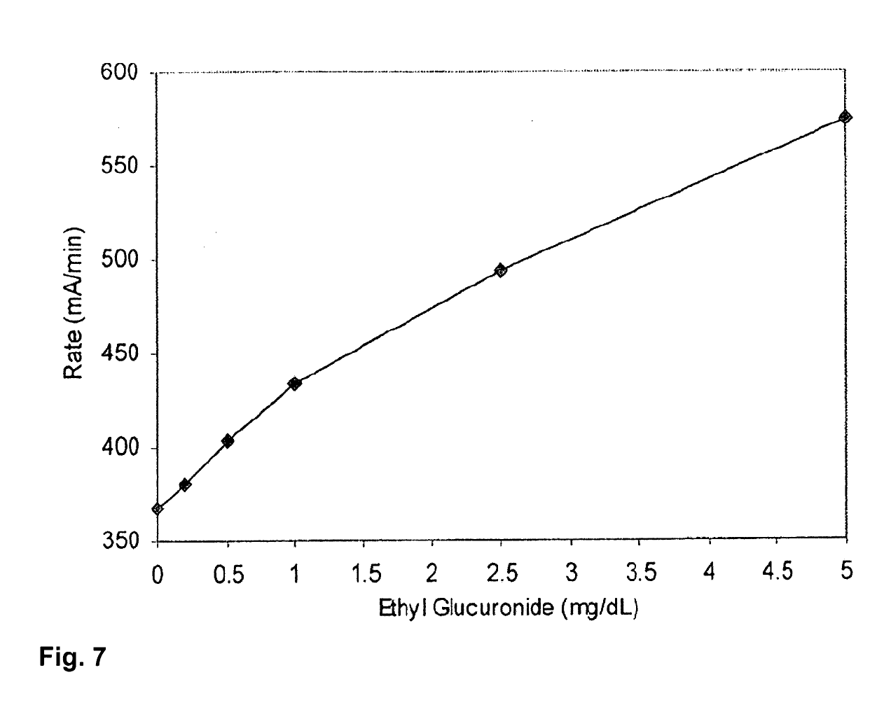

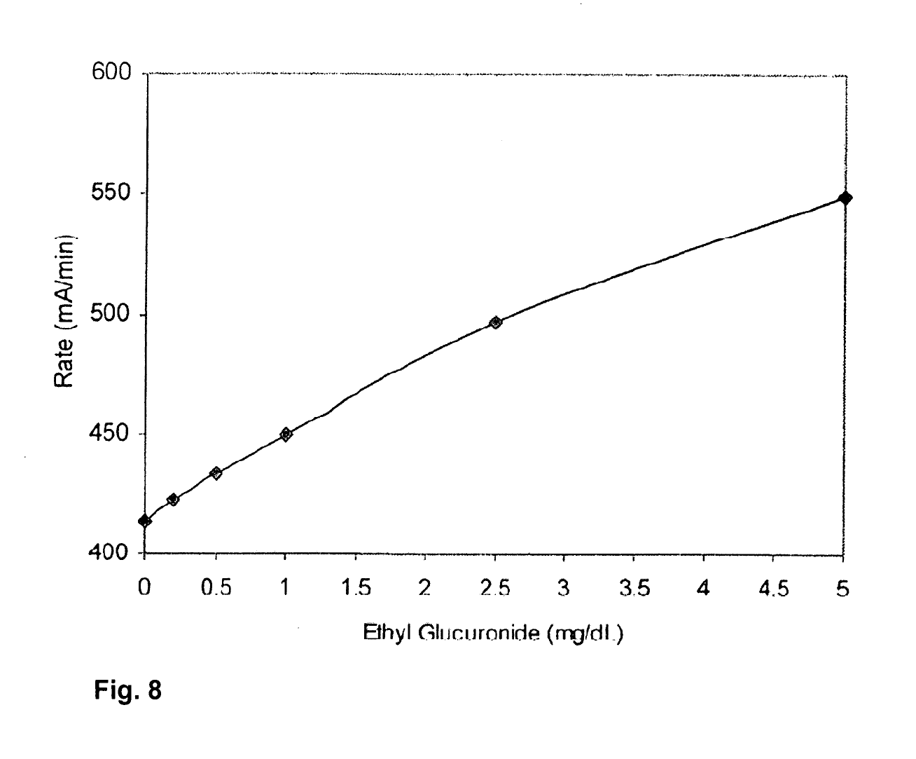

A typical calibration curve for the hybridoma cell line 19D7 using the Hitachi 917 analyzer is shown in FIG. 7, and a typical calibration curve using the Hitachi 717 analyzer is shown in FIG. 8. The limit of detection is approximately 0.05 mg/dL in urine or serum. Run precision for samples within a 0.5-5.0 mg/dL concentration range is 5.4% -11.1%. Run precision for samples within a 0.5-5.0 mg/dL concentration range is 1.7% to 9. 1%. Recovery rates of spiked samples within a concentration range of 0.1-5.0 mg/dL is 93% to 99%.

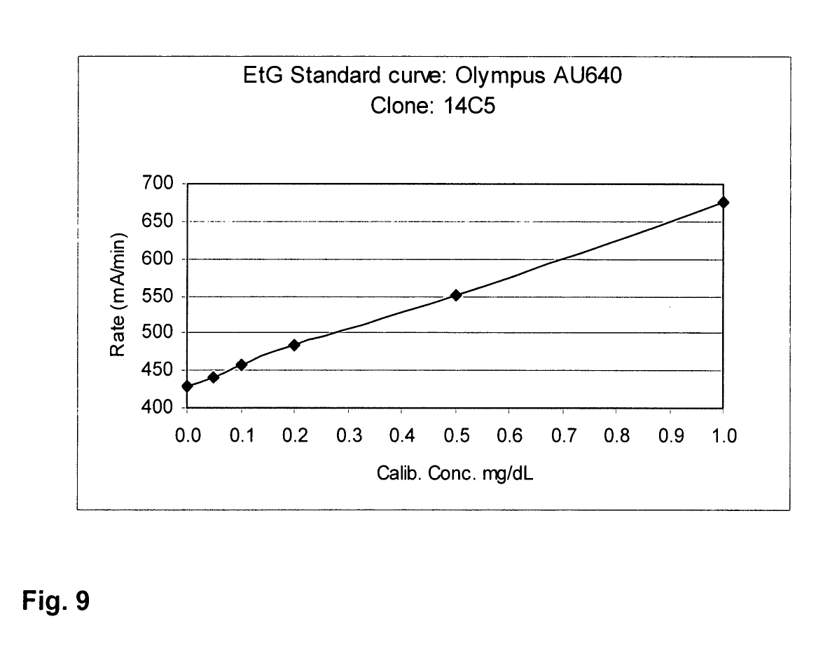

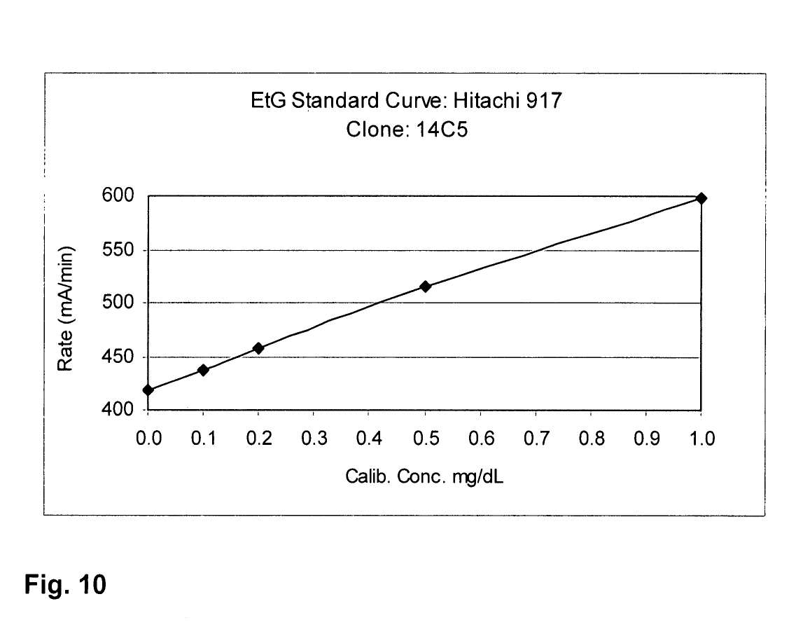

Additionally, another calibration curve for the hybridoma cell line 14C5 using the Olympus AU640 analyzer is shown in FIG. 9, and a typical calibration curve using the Hitachi 917 analyzer is shown in FIG. 10. The concentrations of EtG were 0, 0.05, 0.1, 0.2, 0.5, 1.0 mg/dL. As shown, the limit of detection is substantially lower for the hybridoma cell line 14C5 compared to the hybridoma cell line 19D7.

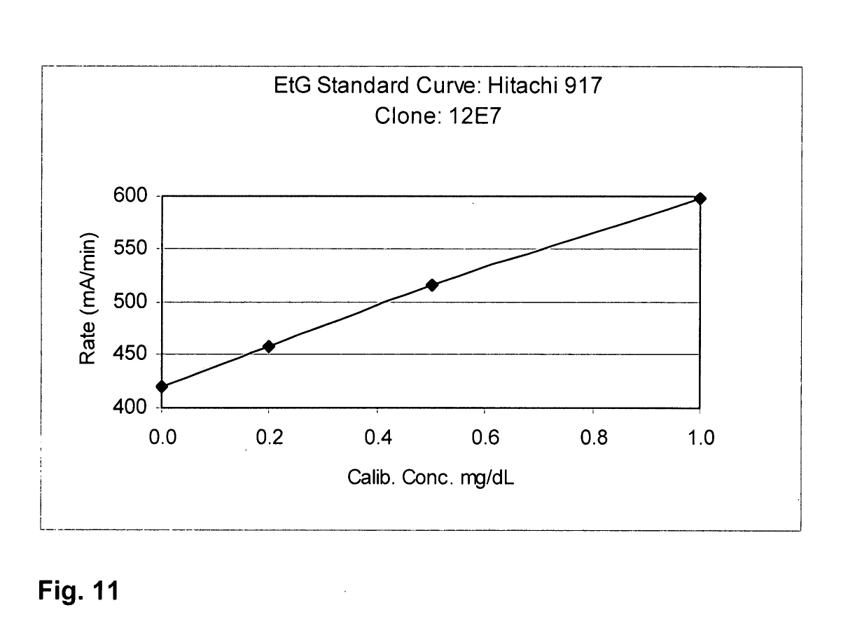

Additionally, another calibration curve for the hybridoma cell line 12E7 using the Hitachi 917 analyzer is shown in FIG. 11. The concentrations of EtG were 0, 0.05, 0.1, 0.2, 0.5, 1.0 mg/dL. As shown, the limit of detection is substantially lower for the hybridoma cell line 12E7 compared to the hybridoma cell line 19D7. Accordingly, the antibodies prepared from the hybridoma cell lines 12E7 and 14C5 provide lower limits of detection for EtG compared to the hybridoma cell line 19D7. Thus, improvements in sensitivity and/or accuracy can be made by selection of hybridoma cell lines.

Specificity of the antibody/immunoassay for EtG was tested by measuring cross-reactivity to other compounds as listed in Table 1. Spiked urine samples were used and all produced negative results (observed concentration <0.05 mg/dL).

TABLE 1

| Compound | Concentration Tested |
|---|---|
| Methyl glucuronide | 100 mg/dL |
| Lorazepam glucuronide | 10 mg/dL |
| Oxazepam glucuronide | 10 mg/dL |
| Temazepam glucuronide | 10 mg/dL |
| D-glucose | 100 mg/dL |
| 1-butanol | 100 mg/dL |
| 2-butanol | 100 mg/dL |

### Example 7

### Homogeneous Competitive Immunoassays

A CEDIA® assay for EtG comprises the following reagents: anti-EtG antibody, polyclonal or monoclonal; P-galactosidase enzyme acceptor (“EA”) reagent comprising EA lyophilized in a buffered saline solution, preferably at a concentration of about 0.118 grams of EA per liter of buffered saline prior to lyophilization, and also, preferably, a preservative such as sodium azide; EA reconstitution buffer, such as Tris, PIPES, HEPES, TES, MOPS or the like; P-galactosidase enzyme donor (“ED”) fragment, preferably ED28, conjugated to EtG, as shown in FIG. 5, lyophilized along with the substrate, and preferably containing a preservative such as sodium azide for extending shelf-life; and ED reconstitution buffer, such as MES, containing a non-ionic detergent (Tween 20, NP-40, etc.) and, preferably, a preservative for extending shelf-life.

The anti-EtG antibody is preferably supplied already mixed in the EA reconstitution buffer. Additionally, a kit may contain calibrators and/or standards. Kit components for performing CEDIA® assays have been generally described in patents cited above and incorporated herein in their entirety.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An ethyl glucuronide analog for use in a process for preparing and/or implementing an immunoassay for detecting ethyl glucuronide in a sample, the analog comprising:
a chemical structure of one of Formula 1, Formula 2, or Formula 3; 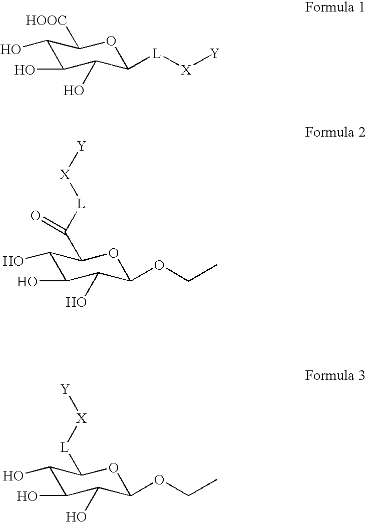
wherein:
L is at least one of the groups O, S, CO, COO, SO₂, CH₂, NH, NIH(CH₂)₂NH, CONH, Ph, or NHCH₂Ph;
X is at least one of a bond between L and Y, an aromatic group, or an aliphatic group; and
Y is selected from the group consisting of alcohol, amine, amide, carboxylic acid, aldehyde, ester, activated ester, aliphatic ester, imidoester, isocyanate, isothiocyanate, anhydride, thiol, thiolactone, diazonium, maleimido, NHS, O—NHS, and a linker derived therefrom coupled with an operative moiety.

2. An analog as in claim 1, wherein X is at least one of a bond between L and Y, a substituted or unsubstituted aromatic or aliphatic group having from 1 to 2 rings, or a saturated or unsaturated, substituted or unsubstituted, and straight or branched chain having from 1 to 20 carbon and/or hetero chain atoms.

3. An analog as in claim 1, wherein the operative moiety coupled to Y is selected from the group consisting of proteins, lipoproteins, glycoproteins, polypeptides, poly(amino acids), polysaccharides, nucleic acids, polynucleotides, teichoic acids, detectable labels, radioactive isotopes, enzymes, enzyme fragments, enzyme donor fragments, enzyme acceptor fragments, enzyme substrates, enzyme inhibitors, coenzymes, fluorescent moieties, phosphorescent moieties, anti-stokes up-regulating moieties, chemiluminescent moieties, luminescent moieties, dyes, sensitizers, particles, microparticles, magnetic particles, solid supports, liposomes, ligands, receptors, hapten radioactive isotopes, albumin, human serum albumin, bovine serum albumin, keyhole limpet hemocyanin, and combinations thereof.

4. An ethyl glucuronide analog for use in a process for preparing an antibody and/or for implementing an immunoassay for detecting ethyl glucuronide in a sample, the analog comprising:
a chemical structure of one of Formula 4, Formula 5, or Formula 6; 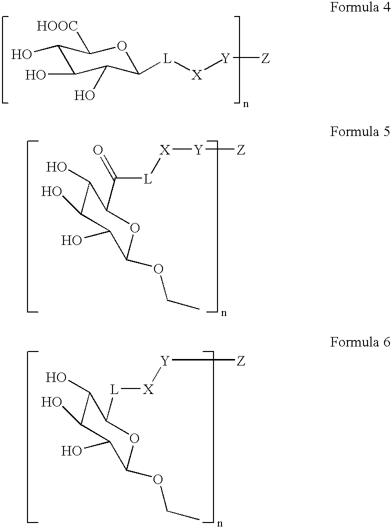
wherein:
n is greater than or equal to about 1 and less than about 1000;
L is at least one of the groups O, S, CO, COO, SO₂, CH₂, NH, NH(CH₂)₂NH, CONH, Ph, or NHCH₂Ph;
X is at least one of a bond between L and Y, a substituted or unsubstituted aromatic or aliphatic group having from 1 to 2 rings, or a saturated or unsaturated, substituted or unsubstituted, and straight or branched chain having from 1 to 20 carbon and/or hetero chain atoms;
Y is selected from the group consisting of alcohol, amine, amide, carboxylic acid, aldehyde, ester, activated ester, aliphatic ester, imidoester, isocyanate, isothiocyanate, anhydride, thiol, thiolactone, diazonium, maleimido, NHS, O—NHS, and a linker derived therefrom; and
Z is an operative moiety.

5. An analog as in claim 4, wherein Z is selected from the group consisting of proteins, lipoproteins, glycoproteins, polypeptides, poly(amino acids), polysaccharides, nucleic acids, polynucleotides, teichoic acids, detectable labels, radioactive isotopes, enzymes, enzyme fragments, enzyme donor fragments, enzyme acceptor fragments, enzyme substrates, enzyme inhibitors, coenzymes, fluorescent moieties, phosphorescent moieties, anti-stokes up-regulating moieties, chemiluminescent moieties, luminescent moieties, dyes, sensitizers, particles, microparticles, magnetic particles, solid supports, liposomes, ligands, receptors, hapten radioactive isotopes, and combinations thereof.

6. An analog as in claim 5, wherein the analog is an immunogen, and Z is at least one of the following:
human serum albumin with n being about 1 to about 35;
bovine serum albumin with n being about 1 to about 35; or
keyhole limpet hemocyanin with n being about 1 to about 500.

7. An analog as in claim 5, wherein the analog is an immunoassay reagent for detecting ethyl glucuronide, and Z is a detectable label.

8. An analog as in claim 7, wherein the detectable label is comprised of an enzyme, enzyme fragment, or enzyme donor fragment.

9. An analog as in claim 8, wherein the enzyme is G6PDH or the enzyme donor fragment is P-galactosidase enzyme donor fragment ED28.

10. An antibody prepared with an immunogen in accordance with the analog as in claim 4, wherein the antibody is an anti-ethyl glucuronide antibody capable of interacting with ethyl glucuronide and the ethyl glucuronide analog.

11. An antibody as in claim 10, wherein the antibody is capable of interacting with ethyl glucuronide in a sample at a concentration of less than or equal to about 0.05 mg/dL and with a cross-reactivity of less than about 1% with at least one of methyl glucuronide, lorazepam glucuronide, oxazepam glucuronide, temazepam flucuronide, D-glucose, 1-butanol, or 2-butanol.

12. An immunoassay system for detecting ethyl glucuronide, the system comprising the anti-ethyl glucuronide antibody of claim 10.

13. An immunoassay system as in claim 12, further comprising the immunoassay reagent of claim 7.

14. A method of detecting ethyl glucuronide in a sample, the method comprising:
obtaining a sample from a subject suspected of consuming ethyl alcohol;
combining an anti-ethyl glucuronide antibody and an ethyl glucuronide analog with the sample to form a first composition, said antibody and analog being free within the first composition and said antibody is capable of binding ethyl glucuronide and the glucuronide analog;
allowing any free ethyl glucuronide from the sample and the free analog to compete for binding with the free antibody; and
detecting binding between the analog and the antibody.

15. A method as in claim 14, wherein the anti-ethyl glucuronide antibody is prepared with an ethyl glucuronide-based immunogen, said immunogen comprising:
a chemical structure of one of Formula 4, Formula 5, or Formula 6; 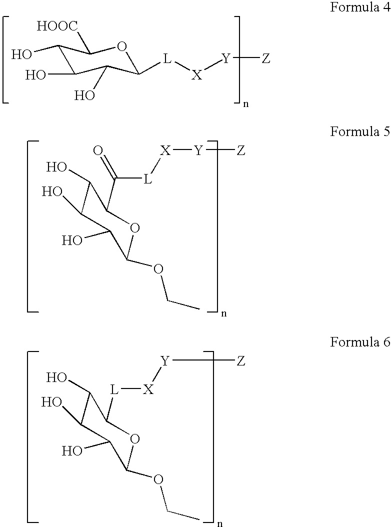
wherein:
n is greater than or equal to about 1 and less than about 1000;
L is at least one of the groups O, S, CO, COO, SO₂, CH₂, NH, NH(CH₂)₂NH, CONH, Ph, or NHCH₂Ph;
X is at least one of a bond between L and Y, a substituted or unsubstituted aromatic or aliphatic group having from 1 to 2 rings, or a saturated or unsaturated, substituted or unsubstituted, and straight or branched chain having from 1 to 20 carbon and/or hetero chain atoms;
Y is selected from the group consisting of alcohol, amine, amide, carboxylic acid, aldehyde, ester, activated ester, aliphatic ester, imidoester, isocyanate, isothiocyanate, anhydride, thiol, thiolactone, diazonium, maleimido, NHS, O—NHS, and a linker derived therefrom; and
Z is an immunogenic operative moiety.

16. A method as in claim 14, wherein the ethyl glucuronide analog is a detectable immunoassay reagent, said immunoassay reagent comprising:
a chemical structure of one of Formula 4, Formula 5, or Formula 6; 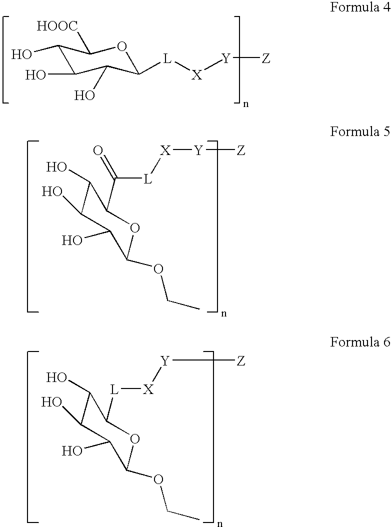
wherein:
n is greater than or equal to about 1 and less than about 1000;
L is at least one of the groups O, S, CO, COO, SO₂, CH₂, NH, NH(CH₂)₂NH, CONH, Ph, or NHCH₂Ph;
X is at least one of a bond between L and Y, a substituted or unsubstituted aromatic or aliphatic group having from 1 to 2 rings, or a saturated or unsaturated, substituted or unsubstituted, and straight or branched chain having from 1 to 20 carbon and/or hetero chain atoms;
Y is selected from the group consisting of alcohol, amine, amide, carboxylic acid, aldehyde, ester, activated ester, aliphatic ester, imidoester, isocyanate, isothiocyanate, anhydride, thiol, thiolactone, diazonium, maleimido, NHS, O—NHS, and a linker derived therefrom; and
Z is a detectable label.

17. A method as in claim 16, wherein the detectable label is comprised of an enzyme, enzyme fragment or enzyme donor fragment.

18. A method as in claim 17, wherein the enzyme is G6PDH or the enzyme donor fragment is β-galactosidase enzyme donor fragment ED28.

19. A method as in claim 14, further comprising:
obtaining the ethyl glucuronide analog, wherein the ethyl glucuronide analog includes an enzyme donor;
combining an enzyme acceptor with the first composition;
combining a substrate with the first composition, wherein the substrate is cleavable by interacting with the enzyme donor and enzyme acceptor; and
detecting enzyme activity.

20. A method as in claim 19, further comprising:
combining a known amount of ethyl glucuronide with the ethyl glucuronide analog and antibody to form a control binding composition;
combining an enzyme acceptor with the control binding composition;
combining a substrate with the control binding composition, wherein the substrate is cleavable by interacting with the enzyme donor and enzyme acceptor;
detecting control enzyme activity; and
determining the amount of ethyl glucuronide present in the sample, wherein a comparison between the enzyme activity and the control enzyme activity is an indication of the amount of ethyl glucuronide present in the sample.

21. A method as in claim 14, further comprising:
obtaining the ethyl glucuronide analog, wherein the ethyl glucuronide analog includes an enzyme;
combining a substrate with the first composition, wherein the substrate is processed by interacting with the enzyme; and
detecting enzyme activity.

22. A method as in claim 21, further comprising:
combining a known amount of ethyl glucuronide with the ethyl glucuronide analog and antibody to form a control binding composition;
combining a substrate with the control binding composition, wherein the substrate is processed by interacting with the enzyme;
detecting control enzyme activity; and
determining the amount of ethyl glucuronide present in the sample, wherein a comparison between the enzyme activity and the control enzyme activity is an indication of the amount of ethyl glucuronide present in the sample.

23. A method as in claim 21, wherein the enzyme is G6PDH.
